(19) United States
(12) Patent Application Publication
(10) Pub. No.: US 2013/0203146 A1
(43) Pub. Date: Aug. 8, 2013

(54) MICROFABRICATED SCAFFOLD STRUCTURES

(71) Applicants: Jackie Y. Ying, Singapore (SG); Tseng-Ming Hsieh, Singapore (SG); Andrew Chwee Aun Wan, Singapore (SG)

(72) Inventors: Jackie Y. Ying, Singapore (SG); Tseng-Ming Hsieh, Singapore (SG); Andrew Chwee Aun Wan, Singapore (SG)

(21) Appl. No.: 13/813,936

(22) PCT Filed: Aug. 3, 2011
(86) PCT No.: PCT/SG2011/000272

§ 371 Date: Apr. 15, 2013

Related U.S. Application Data
(60) Provisional application No. 61/370,166, filed on Aug. 3, 2010.

(51) Int. Cl. C12N 5/00

(52) U.S. Cl. CPC C12N 5/0062
USPC 435/177

(57) ABSTRACT

The present invention relates to a method for producing a three-dimensional scaffold construct comprising encapsulated cells, the method comprising: (a) providing a solution comprising cells, a photoinitiator, and a plurality of units capable of forming polymer chains; (b) providing a photolithography instrument comprising a two-photon laser; and (c) using the instrument to apply the laser to the solution to activate the photoinitiator thereby facilitating polymerisation of said units to form polymer chains, and, cross-linking of the polymer chains; wherein the laser is applied to the solution in three-dimensions in a pre-defined pattern to assemble said construct, and said cells are encapsulated within the assembled construct.

### INCORPORATION BY REFERENCE

This application claims priority from U.S. provisional patent application No. 61/370,166 filed on 3 Aug. 2010, the entire contents of which are incorporated by reference in their entirety.

### TECHNICAL FIELD

The invention relates generally to the field of tissue engineering. More specifically, the invention relates to microfabricated scaffold constructs and methods for their production.

### BACKGROUND

Conventionally, three-dimensional (3D) structures are comprised of multiple layers of cells, obtained either by cell-sheet assembly or by cell-seeding onto a 3D polymer. The thick layers of cells deprive the inner layer of cells from the nutrients and oxygen needed for healthy growth. Even when the constructs are cultured on bioreactors, 100 μm or 4-7 cell layers are the maximum dimensions for a bioreactor to function efficiently (see, for example, Zandonella, (2003), “The beat goes on”, Nature; 421:884-86). In addition, there are other limitations that hinder the construction of 3D scaffolds, one of which is the uneven cell density distribution for cells seeded on acellular 3D scaffolds (see, for example, Tsang and Bhatia, (2004), “Three-dimensional tissue fabrication”, Adv Drug Deliv Rev; 56:1635-47).

This has stimulated research on the use of hydrogel polymers, which render both structural support and high cell density. However, cell patterning within hydrogels involves other issues. For example, in 3D printing, the resolution of patterning is limited to the polymer particle size, and fabrication can only be performed under a narrow set of conditions (such as sterility, temperature and pH). Furthermore, the photopatterning of cell-hydrogel hybrids exposes cells to ultraviolet light, which damages the DNA of the cells (Miller et al. (1996), “The role of ultraviolet light in the induction of cellular DNA damage by a spark-gap lithotripter in vitro”. J Urology; 156:286-90). Microchannels used to grow cells have a depth that renders nutrients diffusion inefficient, thus decreasing the viability of the cells (see, for example, Leclerc et al. (2006), “Guidance of liver and kidney organotypic cultures inside rectangular silicone microchannels”, Biomaterials; 27:4109-19). Despite some progress in obtaining a high cell density for cells seeded on biodegradable scaffolds made of natural or synthetic polymers, the problem of diffusion limitation prevails as nutrients from the culture media are not able to efficiently reach or perfuse the cells attached on the scaffolds.

In view of these and other deficiencies in currently existing techniques, there is a need for new methods of engineering micropatterned three-dimensional constructs for the seeding of cells.

### SUMMARY OF THE INVENTION

The present invention relates to a two-photon technology capable of building high-resolution three-dimensional tissue constructs. The technology provides a simple and flexible method for producing microstructures leading to cell growth in three-dimensional cell culture and tissue engineering.

In a first aspect, the invention provides a method for producing a three-dimensional scaffold construct comprising encapsulated cells, the method comprising:

(a) providing a solution comprising cells to be encapsulated, a photoinitiator, and a plurality of units capable of forming polymer chains;

(b) providing a photolithography instrument comprising a two-photon laser; and

(c) using the instrument to apply the laser to the solution to activate the photoinitiator thereby facilitating polymerisation of said units to form polymer chains, and, cross-linking of the polymer chains;

wherein the laser is applied to the solution in three-dimensions in a pre-defined pattern to assemble said construct, and said cells are encapsulated within the assembled construct.

In a second aspect, the invention provides a method for producing a three-dimensional scaffold construct comprising encapsulated cells, the method comprising:

providing a solution comprising cells to be encapsulated, a photoinitiator, and either or both of:
(a) a plurality of units capable of forming polymer chains,
(b) a plurality of polymer chains;

providing a photolithography instrument comprising a two-photon laser; and

using the instrument to apply the laser to the solution to activate the photoinitiator thereby facilitating polymerisation of said units and/or polymer chains, and cross-linking of said polymer chains;

wherein the laser is applied to the solution in three-dimensions in a pre-defined pattern to assemble said construct, and said cells are encapsulated within the assembled construct.

In one embodiment of the first and second aspects, the scaffold construct is assembled according to a three dimensional computer assisted design (CAD) image that is read by said photolithography instrument.

In one embodiment of the first and second aspects, the cells are encapsulated during cross-linking of the polymer chains in three dimensions.

In one embodiment of the first and second aspects, the cells are encapsulated by cross-linking of the polymer chains in three dimensions.

In one embodiment of the first and second aspects, the laser emits energy in the infrared region.

In one embodiment of the first and second aspects, the cells comprise human umbilical vascular endothelial cells (HUVEC).

In one embodiment of the first and second aspects, the cells comprise hepatocytes.

In one embodiment of the first and second aspects, the cells comprise stem cells.

In one embodiment of the first and second aspects, the construct comprises more than one type of polymer chain.

In one embodiment of the first and second aspects, the unit is monomer of a resin polymer.

In one embodiment of the first and second aspects, the unit is a fibrillar protein.

In one embodiment of the first and second aspects, the fibrillar protein is fibrinogen.

In one embodiment of the first and second aspects, the photoinitiator is ruthenium II trisbipyridyl chloride [RuII(bpy)₃]²⁺, and the solution comprises an oxidising agent.

In one embodiment of the first and second aspects, the oxidising agent is sodium persulfate.

In one embodiment of the first and second aspects, the construct is ring-shaped.

In one embodiment of the first and second aspects, the pores are between about 1 μm and about 50 μm in width or diameter.

In one embodiment of the first and second aspects, the pores are between about 1 μm and about 10 μm in width or diameter.

In one embodiment of the first and second aspects, the method further comprises washing the construct to substantially remove non-crosslinked polymer chains and non polymerised units.

In one embodiment of the first and second aspects, the polymer chains are biodegradable.

In one embodiment of the first and second aspects, the solution further comprises a bioactive component.

In one embodiment of the first and second aspects, the cells are in the solution at a concentration of between about 1×10⁶/ml and about 1×10⁷/ml.

In one embodiment of the first and second aspects, the method further comprises seeding additional cells to the construct after completion of said polymerization and cross-linking.

In one embodiment of the first and second aspects, the ring-shaped construct has a diameter of about 400 μm, and a thickness of about 100 μm.

In a third aspect, the invention provides a scaffold construct produced in accordance with the method of the first aspect or the second aspect.

### BRIEF DESCRIPTION OF THE DRAWINGS

Preferred embodiments of the invention will now be described, by way of example only, with reference to the accompanying figures wherein:

FIG. 1 is a graph illustrative of degradation of cross-linked fibrin in media containing ( [symbol] ) Tris buffer only (control), and (▪) 0.1 μg/ml, (D) 1.0 μg/ml, () 10 μg/ml, (▾) 50 μg/ml over 24 days.

FIG. 2 provides light microscopy images of HUVECs seeded on fibrin surface after (A) 24 h and (B) 48 h. Cells were stained with the Live/Dead® assay.

FIG. 3 is a graph illustrative of the effect of [Rull(bpy)₃]²⁺ concentration on the viability of HUVECs. Absorbance of the MTT assay was determined at 490 nm.

FIG. 4 provides light microscopy images of (A, C) the fibrin constructs with cells stained with (A, C) the Live/Dead® assay and (B, D) the EthD-1 component of the Live/Dead® assay. Cells in the background represent those that were not washed away and remained attached onto the cover slip. (A) Images of four scanned devices on a cover slip showing rings of live cells grown on the fibrin constructs. (B) Image taken from the channel to view EthD-1 fluorescence in (A). The fibrin constructs display auto-fluorescence, giving the false appearance of a ring of dead cells. (C) Magnified image of (A) showing one of the constructs. (D) Image taken from channel to view EthD-1 fluorescence in (C), showing the auto-fluorescence of the fibrin construct.

FIG. 5 provides (A) Bright-field image of the fibrin construct. HUVECs encapsulated within the scaffold were slightly visible. The brown lines depict the way the laser beam scans the fibrinogen mixture; and light microscopy images of HUVECs in the fibrin construct stained by the Live/Dead® assay (B) immediately after scanning, and after (C) 1 day and (D) 5 days of culture. Image (C) illustrated fast cell attachment and spreading; the cells were elongated along the curvature of the device. Image (D) was focused at a certain focal plane to best display the ring of cells on the inner and outer boundaries of the scaffold. Scale bar=100 μm.

FIG. 6 provides scanning electron microscope images of (A) fibrin constructs on a cover slip after freeze drying, illustrating the 3D structure of the constructs; and (B) Image of one construct with higher magnification.

FIG. 7 provides fluorescent microscopy images showing (A) fibrin constructs without HUVECs, showing that fibrin absorbed the EthD-1 dye of the Live/Dead® assay and appeared red; and (B) the intensity of the red dye was greatly reduced after washing with PBS.

FIG. 8 provides confocal microscopy images of fibrin constructs with HUVECs after 5 days of culture.

FIG. 9 shows a computer assisted design (CAD) of a 3D microstructured scaffold (2.5 mm×2.5 mm×2.5 mm) referred to in Example 2 of the specification.

FIG. 10 shows an absorbance spectrum of SI10 photopolymer. Polymer is near transparent in the UV-vis range.

FIG. 11 shows micrsoscopy images of 3D microstructures formed by TPLSP as described in Example 2: (A) side view and (B) top view.

FIG. 12 shows fluorescence microscopy images of HepG2 with GFP attached onto grafted 3D polymeric scaffold at (A) lower and (B) higher magnification. (C) Confocal image of the 3D scaffold with seeded HepG2.

FIG. 13 provides microscopy images showing immunofluorescence labeling of hepatocyes cultured within the 3D polymeric scaffold on Day 4. Hepatocytes were detached from the scaffolds and placed on a glass slide prior to staining. Nuclei, albumin and fibronectin were stained with DAPI, FITC and Texas Red, respectively.

FIG. 14 provides graphs depicting liver-specific functions of hepatocytes cultured within 3D microstructured scaffolds and on 2D polymeric substrates, as assessed by (A) albumin secretion and (B) urea synthesis over a 6-day culture period (*p<0.05).

### DEFINITIONS

As used in this application, the singular form “a”, “an” and “the” include plural references unless the context clearly dictates otherwise. For example, the term “a polymer” also includes a plurality of polymers.

As used herein, the term “comprising” means “including.” Variations of the word “comprising”, such as “comprise” and “comprises,” have correspondingly varied meanings. Thus, for example, a construct “comprising” a given polymer type may consist exclusively of that polymer type or may include one or more additional polymer types.

As used herein, the term “photopolymer” encompasses a polymer, and monomer units capable of assembling into a polymer, that can be made to polymerise and/or cross-link, upon exposure to a form of electromagnetic radiation (e.g. infrared light, visible light, ultraviolet light, X-rays, gamma rays). The polymerizing and/or cross-linking may occur spontaneously upon exposure to electromagnetic radiation, or may require (or be enhanced by) the presence of one or more additional compounds (e.g. a catalyst, or a photoinitiator).

As used herein, a “photoinitiator” is a molecule that upon absorption of light at a specific wavelength produces one or more reactive species capable of catalyzing polymerization, cross-linking and/or curing reactions.

As used herein, “two-photon laser scanning photolithography” refers to the use of two photon excitation of fluorescence in laser scanning photolithography. “Two-photon excitation” occurs when a molecule (or fluorophore) is excited via near simultaneous or simultaneous absorption of two photons of identical or different frequencies, which excites the molecule/fluorophore from one state (usually the ground state) to a higher energy electronic state. The energy difference between the involved lower and upper states of the molecule/fluorophore is substantially equal to, or equal to, the sum of the energies of the two photons.

It will be understood that use of the term “about” herein in reference to a recited numerical value includes the recited numerical value and numerical values within plus or minus ten percent of the recited value.

It will be understood that use of the term “between” herein when referring to a range of numerical values encompasses the numerical values at each endpoint of the range. For example, a polymer of between 10 monomers and 20 monomers in length is inclusive of a polymer of 10 monomers in length and a polymer of 20 monomers in length.

Any description of prior art documents herein, or statements herein derived from or based on those documents, is not an admission that the documents or derived statements are part of the common general knowledge of the relevant art.

For the purposes of description all documents referred to herein are hereby incorporated by reference in their entirety unless otherwise stated.

### DETAILED DESCRIPTION

Many current tissue engineering protocols require the seeding of cells onto a scaffold. When the scaffold design becomes smaller in dimensions and more complex, difficulties are encountered in seeding cells into tiny pores because of diffusion limitation. This has a negative impact on the ability of bioreactors to supply sufficient nutrients and oxygen to the growing tissue. For example, while human heart muscle is up to 2 cm thick, growth in a bioreactor typically stops once the tissue is approximately 100 μm, or 4-7 cell layers for cell sheet technology. Although cell/gel printing is a bottom-up technology capable of constructing a scaffold layer by layer, droplet printing to date fails to provide a high cell density and the fine structure needed for advanced tissue engineering.

The present invention provides methods for producing high-resolution three-dimensional (3D) tissue scaffolding constructs. The methods facilitate the encapsulation of cells during formation of the microfabricated structures thus providing a means of bypassing the cell seeding process. More specifically, the invention provides a laser scanning photolithography technique that can be used to excite crosslinkable molecules of polymeric compounds to form a dense 3D polymer network in a specific target pattern. Live cells may be encapsulated during construction of the 3D network, whilst retaining their viability under laser scanning. In this manner, a mixture of polymeric compounds and live cells can be used to construct a 3D microstructured scaffold comprising encapsulated cells.

The present invention also provides high-resolution three-dimensional (3D) tissue scaffolding constructs. The scaffolding constructs can be fabricated in a manner that enables entrapment of cells at high density and viability. Moreover, the constructs can provide mechanical support and directed cell spreading according to their shape and curvature.

### Polymers

The present invention provides scaffolds constructed from polymers and methods for their production.

Without placing any particular limitation on the type of polymers that may be used in a method or construct of the present invention, certain characteristics may be desirable. For example, the polymers may be biocompatible (i.e. non-toxic), non-immunogenic, have a capacity to act as adhesive substrates for cells, promote cell growth, and/or allow the retention of differentiated cell function.

Additionally or alternatively, the polymers may comprise one or more physical characteristics allowing for mechanical strength, large surface to volume ratios, and/or straightforward processing into desired shape configurations.

A scaffold constructed from a polymer in accordance with the methods of the invention may be rigid enough to maintain the desired shape under in vivo conditions.

A polymer used in a method or construct of the present invention may be biodegradable or substantially biodegradable. Preferably, the degraded products of the polymer are biocompatible.

The polymer may be a homopolymer or a copolymer.

The polymer may be synthetic or natural.

Non-limiting examples of potentially suitable synthetic polymers include polyesters (e.g. Poly(glycolic acid), Poly(1-lactic acid), Poly(d,l-lactic acid), Poly(d,l-lactic-co-glycolic acid), Poly(capro lactone), Poly(propylene fumarate), poly(p-dioxanone), poly(trimethylene carbonate), and their copolymers, polyanhydrides (e.g. Poly [1,6-bis(carboxyphenoxy)hexane]), Poly(phosphoesters) (e.g. poly(bis(hydroxyethyl), terephthalate-ethyl, ortho-phosphate/terephthaloyl chloride), poly(ortho esters) (e.g. Alzamer®), polycarbonates (e.g. Tyrosine-derived polycarbonate), polyurethanes (e.g. Polyurethane based on LDI and poly(glycolide-co-γ-caprolactone)), and polyphosphazenes (e.g. ethylglycinate polyphosphazene).

Non-limiting examples of potentially suitable natural polymers include those derived from proteins such as collagen; fibrin, gelatin, albumin and polysaccharides such as cellulose, hyaluronate, chitin, glycosaminoglycans (e.g. hyaluronic acid), proteoglycans (e.g. chondroitin sulphate, heparin), fibronectin, laminin, and alginate.

In certain embodiments, the polymer may comprise proteins. The proteins may be fibrillar proteins. Non-limiting examples of suitable fibrillar proteins include collagen, elastin, fibrinogen, fibrin, albumin and gelatin.

A polymer used in a method or construct of the present invention may exist as a polymer in its natural state. Such polymers may be further polymerised and/or cross-linked with other polymers.

Additionally or alternatively, a polymer used in a method or construct of the present invention may be prepared from monomer units using any suitable technique known in the art. Polymer chains may also be further polymerised by the addition of further monomer unit(s) and/or by linking with other polymer chains.

In certain embodiments, monomer units and/or separate polymer chains may be linked together using a suitable polymerising agent. Polymerisation agents and methods for their use are well known to those of skill in the art. Non-limiting examples of potentially suitable polymerisation agents include diisocyanates, peroxides, diimides, diols, triols, epoxides, cyanoacrylates, enzymes (e.g. polymerases) and the like.

A polymer used in a method or construct of the present invention may be cross-linked to form a polymer network. The polymer networks may be two-dimensional or three-dimensional. Potentially suitable cross-linking agents include, but are not limited to, genipin, glutaraldehyde, carbodiimides (e.g. EDC), imidoesters (e.g. dimethyl suberimidate), N-Hydroxysuccinimide-esters (e.g. BS3), divinyl sulfone, epoxides, imidazole, sugars (e.g. pentoses or hexoses).

By way of non-limiting example only, a fibrin polymer may be formed from fibrinogen monomer precursors in the presence of a serine protease (e.g. thrombin) to initiate the spontaneous aggregation of fibrin monomers into a nanofibrous network. Calcium ions and factor XIII (a transglutaminase) may then be used to covalently crosslink the fibrin polymers.

A polymer used in a method or construct of the present invention may be a “photopolymer”. As used herein, the term “photopolymer” encompasses a polymer, and monomer units capable of assembling into a polymer, that can be made to polymerise and/or cross-link, upon exposure to a form of electromagnetic radiation (e.g. infrared light, visible light, ultraviolet light, X-rays, gamma rays). The polymerizing and/or cross-linking may occur spontaneously upon exposure to electromagnetic radiation, or may require (or be enhanced by) the presence of one or more additional compounds (e.g. a catalyst, or a photoinitiator).

Any type of photopolymer may be used in a method or construct of the present invention. Suitable photopolymers may include, but are not limited to, resins (e.g. epoxy resins, acrylate resins, Accura® SI 10), dimethacrylate polymers, poly(propylene fumarate) (PPF), blends of PPF and diethyl fumarate (DEF), photopolymerized poly(ethylene glycol) (PEG), 2-hydroxyethyl methacrylate (HEMA), poly(ethylene glycol)diacrylate (PEGDA), and the like.

A photopolymer used in a method or construct of the present invention may be induced to polymerize, cross-link and/or cure in the presence of a photoinitiator. As used herein, a “photoinitiator” is a molecule that upon absorption of light at a specific wavelength produces one or more reactive species capable of catalyzing polymerization, cross-linking and/or curing reactions. For example, the photoinitiator may be water-compatible and act on molecules containing an acrylate or styrene group (e.g. Irgacure 2959, 184, and 651; VA-086; or V-50). The photoinitiator may be a chromophore. Other non-limiting examples of suitable photoinitiators include ruthenium II trisbipyridyl chloride [RuII(bpy)₃]²⁺, 2,2-dimethoxy-2-phenyl)-acetophenone (Irgacure 651) and 2-photon sensitive chromophore (AF240).

### Laser Scanning

A polymer used in a method or construct of the present invention, may itself be polymerised (i.e. formed) and/or cross-linked to other polymers using energy provided by a laser. In some embodiments, the laser may be a multi-photon or two-photon laser. In preferred embodiments, the laser is a two-photon laser. The laser may be provided as a component of a laser-scanning microscope. For example, a two photon laser may be provided as a component of a two photon laser-scanning microscope.

In preferred embodiments, a polymer used in a method or construct of the present invention may be polymerised and/or cross-linked with other polymers using two-photon laser scanning photolithography. “Two-photon laser scanning photolithography” as used herein refers to the use of two photon excitation of fluorescence in laser scanning photolithography. As known to those of skill in the field, “two-photon excitation” occurs when a molecule (or fluorophore) is excited via near simultaneous or simultaneous absorption of two photons of identical or different frequencies, which excites the molecule/fluorophore from one state (usually the ground state) to a higher energy electronic state. The energy difference between the involved lower and upper states of the molecule/fluorophore is substantially equal to, or equal to, the sum of the energies of the two photons. The high intensity illumination necessary for two-photon excitation is generally achieved within the focal volume. As the laser focal point is the location along the optical path where the two-photon excitation occurs, photoreactive processes such as polymerisation and/or polymer crosslinking may be confined to the microscaled focal volume.

When two-photon excitation is applied in laser-scanning microscopy a diffraction-limited volume (at a focal point) may be illuminated with high intensity light at twice the excitation wavelength. The high intensity may enable the virtually simultaneous arrival of two photons to raise an electron to an elevated state. The high intensity illumination may be attained by focusing a beam from a high energy pulsed laser delivering bursts of about 100 femtosecond to 1-2 picosecond pulses at high frequencies (e.g. 100 MHz).

In preferred embodiments of the present invention two-photon laser scanning photolithography may be used for the generation of porous three-dimensional scaffold constructs.

Non-limiting examples of suitable lasers that may be used for two-photon polymerisation include two photon Titanium/Sapphire lasers, femtosecond infrared lasers, and the like.

In certain embodiments, the laser utilised is ported to a suitable microscope such as, for example, a confocal microscope.

Preferably, the laser is provided as a component of photolithography instrument capable of reading a CAD image of the three-dimensional scaffold construct.

The present invention contemplates the use of “CAD” (computer-aided design) in the generation of scaffold constructs of the present invention. CAD may be used, for example, to direct polymerisation and/or crosslinking of a sample using a laser (e.g. a two-photon laser) and thereby manufacture three-dimensional constructs. As used herein, the term “CAD” includes all manner of computer aided design systems, including pure CAD systems, CAD/CAM systems, and the like, provided that such systems are used at least in part to develop or process a model of a three-dimensional scaffold construct of the present invention. Non-limiting examples include Solidworks (Solidworks Corp.) and LSM software (Zeiss).

In certain embodiments, scaffold constructs are generated using a two-photon laser scanning photolithography system is utilising a microscope with an air lens. The air lens may extend the scan height attainable in comparison to a system utilising an oil lens, thus leading to a greater scan volume. The air lens may also minimise contamination of the sample or system by alleviating the need to use oil.

By way of non-limiting example only, a three-dimensional scaffold construct of the present invention may be constructed by preparing a sample comprising photopolymers and/or monomer units thereof. The sample, may comprise one or more photoinitiators (see, for example, those described in the section above entitled “Polymers”) and/or one or types of cells (see, for example, those described in the section below entitled “Encapsulated Cells”). Polymerisation and/or crosslinking of the sample may be initiated by scanning a two-photon laser in a given x-y plane and/or a given z plane. The laser may be tuned at an appropriate wavelength, such as, for example, a wavelength in the infrared range (e.g. near infrared). The scanning may be performed in a pre-defined pattern in the plane to affect highly localised polymerisation and/or cross-linking of polymer chains in the sample. The laser may be scanned across additional plane(s) in the same or different patterns, thereby facilitating further polymerisation and/or cross-linking of sample and the generation of a three-dimensional scaffold structure. Unpolymerised and uncrosslinked material may be removed from the construct by washing with a suitable reagent (e.g. phosphate buffered saline, culture media).

### Encapsulated Cells

Although not necessarily a requirement, scaffold constructs of the present invention may comprise encapsulated cells. Preferably, the encapsulated cells are live/viable cells.

Many tissue engineering protocols require the seeding of cells onto a pre-fabricated scaffold. However, in many cases it is difficult to seed scaffolds with small-dimension and/or complex pore systems due to diffusion limitation. Although cell/gel printing may be used to drop cell aggregates in sequential layers of a gel, this technique fails to provide a high cell density and high resolution platform.

The methods of the present invention circumvent these problems by allowing for the encapsulation of cells throughout the scaffold during polymerisation and cross-linking of polymer chains. The scaffold constructs of the present invention therefore need not necessarily be seeded with cells post-assembly, and there is no restriction for the cells to be printed into sequential layer(s) of the construct.

In accordance with the present invention, cells may be encapsulated in a scaffold construct by mixing the cells with the material to be polymerised and/or cross-linked prior to forming the scaffold. Polymerisation and/or cross-linking of polymers may then be performed as described herein, resulting in the encapsulation of cells in the construct.

Any given cell type(s) may be encapsulated in the scaffold constructs, including mixtures of different cell types.

Non-limiting examples of cell types that may be encapsulated in the scaffold constructs include human umbilical vascular endothelial cells (HUVEC), embryonic stem cells, adult stem cells, blast cells, cloned cells, placental cells, keratinocytes, basal epidermal cells, urinary epithelial cells, salivary gland cells, mucous cells, serous cells, von Ebner's gland cells, mammary gland cells, lacrimal gland cells, ceruminpus gland cells, eccrine sweat gland cells, apocrine sweat gland cells, MpH gland cells, sebaceous gland cells, Bowman's gland cells, Brunner's gland cells, seminal vesicle cells, prostate gland cells, bulbourethral gland cells, Bartholin's gland cells, Littre gland cells, uterine endometrial cells, goblet cells of the respiratory or digestive tracts, mucous cells of the stomach, zymogenic cells of the gastric gland, oxyntic cells of the gastric gland, insulin-producing P cells, glucagon-producing a cells, somatostatin-producing DELTA cells, pancreatic polypeptide-producing cells, pancreatic ductal cells, Paneth cells of the small intestine, type II pneumocytes of the lung, Clara cells of the lung, anterior pituitary cells, intermediate pituitary cells, posterior pituitary cells, hormone secreting cells of the gut or respiratory tract, thyroid gland cells, parathyroid gland cells, adrenal gland cells, gonad cells, juxtaglomerular cells of the kidney, macula densa cells of the kidney, peri polar cells of the kidney, mesangial cells of the kidney, brush border cells of the intestine, striated ducted cells of exocrine glands, gall bladder epithelial cells, brush border cells of the proximal tubule of the kidney, distal tubule cells of the kidney, conciliated cells of the ductulus efferens, epididymal principal cells, epididymal basal cells, hepatocytes, fat cells, type I pneumocytes, pancreatic duct cells, nonstriated duct cells of the sweat gland, nonstriated duct cells of the salivary gland, nonstriated duct cells of the mammary gland, parietal cells of the kidney glomerulus, podocytes of the kidney glomerulus, cells of the thin segment of the loop of Henle, collecting duct cells, duct cells of the seminal vesicle, duct cells of the prostate gland, vascular endothelial cells, synovial cells, serosal cells, squamous cells lining the perilymphatic space of the ear, cells lining the endolymphatic space of the ear, choroid plexus cells, squamous cells of the pia-arachnoid, ciliary epithelial cells of the eye, corneal endothelial cells, ciliated cells having propulsive function, ameloblasts, planum semilunatum cells of the vestibular apparatus of the ear, interdental cells of the organ of Corti, fibroblasts, pericytes of blood capillaries, nucleus pulposus cells of the intervertebral disc, cementoblasts, cementocytes, odontoblasts, odontocytes, chondrocytes, osteoblasts, osteocytes, osteoprogenitor cells, hyalocytes of the vitreous body of the eye, stellate cells of the perilymphatic space of the ear, skeletal muscle cells, heart muscle cells, smooth muscle cells, myoepithelial cells, red blood cells, platelets, megakaryocytes, monocytes, connective tissue macrophages, Langerhan's cells, osteoclasts, dendritic cells, microglial cells, neutrophils, eosinophils, basophils, mast cells, plasma cells, helper T cells, suppressor T cells, killer T cells, killer cells, rod cells, cone cells, inner hair cells of the organ of Corti, outer hair cells of the organ of Corti, type I hair, cells of the vestibular apparatus of the ear, type II cells of the vestibular apparatus of the ear, type II taste bud cells, olfactory neurons, basal cells of olfactory epithelium, type I carotid body cells, type II carotid body cells, Merkel cells, primary sensory neurons specialised for touch, primary sensory neurons specialised for temperature, primary neurons specialised for pain, proprioceptive primary sensory neurons, cholinergic neurons of the autonomic nervous system, adrenergic neurons of the autonomic nervous system, peptidergic neurons of the autonomic nervous system, inner pillar cells of the organ of Corti, outer pillar cells of the organ of Corti, inner phalangeal cells of the organ of Corti, outer phalangeal cells of the organ of Corti, border cells, Hensen cells, supporting cells of the vestibular apparatus, supporting cells of the taste bud, supporting cells of the olfactory epithelium, Schwann cells, satellite cells, enteric glial cells, neurons of the central nervous system, astrocytes of the central nervous system, oligodendrocytes of the central nervous system, anterior lens epithelial cells, lens fiber cells, melanocytes, retinal pigmented epithelial cells, iris pigment epithelial cells, oogonium, oocytes, spermatocytes, spermatogonium, ovarian follicle cells, Sertoli cells, and thymus epithelial cells, hepatocarcinoma, or combinations thereof, or cell lines derived therefrom.

In embodiments where the scaffold construct is intended for implantation to a given subject, the encapsulated cells may be autologous, allogeneic or xenogeneic to the intended recipient.

The number of cells encapsulated in a given scaffold construct will generally depend on factors such as the dimensions of the construct along with the size and morphology of the cells utilised. Preferably, the scaffold constructs comprise a high density of cells, although the density of cells will depend on the particular application.

In certain embodiments, the scaffold construct is generated by polymerising and/or cross-linking a solution comprising cells at a concentration of between about 50 million and 200 million cells/ml, between about 100 million and 200 million cells/ml, between about 100 million and 150 million cells/ml, between about 1 million cells/ml and about 50 million cells/ml, or between about 1 million cells/ml and about 10 million cells/ml.

In addition to encapsulated cells, scaffold constructs of the invention may comprise other bioactive components. Non-limiting examples of bioactive components include proteins (e.g. extracellular matrix proteins such as collagen, elastin, pikachurin; cytoskeletal proteins such as actin, keratin, myosin, tubulin, spectrin; plasma proteins such as serum albumin; cell adhesion proteins such as cadherin, integrin, selectin, NCAM; and enzymes); neurotransmitters (e.g. serotonin, dopamine, epinephrine, norepinephrine, acetylcholine); angiogenic factors (e.g. angiopoietins, fibroblast growth factor, vascular endothelial growth factor, matrix metalloproteinase enzymes); amino acids; galactose ligands; nucleic acids (e.g. DNA, RNA); drugs (antibiotics, anti-inflammatories, antithrombotics, and the like); polysaccharides; proteoglycans; hyaluronate; cross-linkers such as factor XIII; lysyloxidase; anticoagulants; antioxidants; cytokines (e.g. interferons (IFN), tumor necrosis factors (TNF), interleukins, colony stimulating factors (CSFs)); hormones or growth factors (e.g. insulin, insulin-like growth factor, epidermal growth factor, oxytocin, osteogenic factor extract (OFE), epidermal growth factor (EGF), transforming growth factor (TGF), platelet derived growth factor (PDGF-AA. PDGF-AB, PDGF-BB), acidic fibroblast growth factor (FGF), basic FGF, connective tissue activating peptides (CTAP), thromboglobulin, erythropoietin (EPO), and nerve growth factor (NGF)); or combinations thereof.

The additional bioactive components may be obtained from any source (e.g. humans, other animals, microorganisms). For example, they may be produced by recombinant means or may be extracted and purified directly from a natural source.

Although not specifically required, scaffold constructs comprising encapsulated cells and/or other bioactive components may optionally be seeded with further additional cells after their construction.

In preferred embodiments of the invention, cells may be encapsulated in a scaffold construct generated by two-photon laser scanning photolithography as described in the section above entitled “Laser scanning”. This methodology may be used to allow the fabrication of scaffold constructs in submicron resolution comprising encapsulated cells at high density and viability.

By way of non-limiting example only, a solution comprising fibrinogen, an oxidising agent (e.g. sodium persulfate) and a suitable photoinitiator (e.g. [Rull(bpy)₃]²⁺) may be mixed with a desired cell type (e.g. HUVEC) at an appropriate cell density. Two-photon laser scanning photolithography may be used generate a porous three-dimensional microstructured scaffold comprising encapsulated cells. The laser scanning process may use infrared irradiation to photoexcite the photinitiator in the solution which may minimise any potential ill effects on the cells which do not absorb infrared wavelength radiation. Unpolymerised/uncrosslinked material may be removed from the newly-formed construct by rinsing with a suitable reagent (e.g. cell culture media).

Scaffold constructs of the present invention comprising encapsulated cells may be cultured to promote growth/development and/or induce functionality of encapsulated cells. Apart from general considerations such as pH, temperature, oxygen, nutrients and osmotic pressure, specific requirements such as growth factors, cytokines, chemokines, specific metabolites/nutrients, and chemical/physical stimuli may also be required. A bioreactor may be used to simulate a physiological environment to promote the growth and differentiation of encapsulated cells.

The viability and function of encapsulated cells may be determined using standard techniques known in the art (e.g. Live/Dead assay, microscopy, ELISA and other assays capable of measuring the secretion of cellular factors, cell staining, cell marker phenotyping etc.).

### Scaffold Constructs

A scaffold construct of the present invention may be fabricated in the form of a gel, sleeve, cuff, sponge, membrane, cube, ring, circle, tube, sheet or any other shape useful in biological applications.

In embodiments where the construct is circular or ring-shaped, the diameter of the construct may be less than 500 μm, less than 400 μm, about 400 μm, less than 300 μm, less than 250 μm, less than 150 μm, or less than 100 μm.

In embodiments where the construct is ring-shaped, the height (thickness) of the construct may be less than 300 μm, less than 250 μm, less than 150 μm, or less than 100 μm, or about 100 μm.

A ring-shaped construct may have a diameter of about 400 μm and a height (thickness) of about 100 μm.

In other embodiments, the height of the construct may be less than 5000 μm, less than 4000 μm, less than 3000 μm, less than 2000 μm, less than 1500 μm, less than 1000 μm, less than 500 μm, less than 400 μm, less than 300 μm, less than 200 μm, less than 150 μm, or less than 100 μm.

In other embodiments, the width of the construct may be less than 5000 μm, less than 4000 μm, less than 3000 μm, less than 2000 μm, less than 1500 μm, less than 1000 μm, less than 500 μm, less than 400 μm, less than 300 μm, less than 200 μm, less than 150 μm, or less than 100 μm.

A cube-shaped construct may have a height, width and depth of about 2500 μm. The cube may have a pitch. The pitch size may be about 250 μm.

Scaffold constructs of the invention may be porous. The porosity of the construct is preferably of a size that allows the migration of components (e.g. cells, proteins, growth factors, nutrients, and/or cellular wastes) within and/or through the construct. In some embodiments, the constructs may comprise pores of between about 100 μm and about 1000 μm in width or diameter, between about 100 μm and about 500 μm in width or diameter, between about 10 μm and about 100 μm in width or diameter, between about 1 μm and about 100 μm in width or diameter, between about 1 μm and about 50 μm in width or diameter, less than about 100 μm in width or diameter, or less than about 90 μm, 80 μm, 70 μm, 60 μm, 50 μm, 40 μm 30 μm, 20 μm, 15 μm, 10 μm or 5 μm in width or diameter. In some embodiments, the constructs may comprise substantially circular pores of less than about 70 μm in diameter, less than about 60 μm in diameter, less than about 50 μm, 40 μm, 30 μm, 20 μm, 15 μm, 12 μm, 10 μm, or 5 μm in diameter, or about 10 μm in diameter.

Physicochemical properties of scaffold constructs of the present invention may be evaluated (and compared to those of untreated raw materials if so desired) using techniques such as MRI analysis, microscopy, and other analytical tools known in the art.

In certain embodiments, the scaffold construct may be coated with a substance to enhance the binding of one or more biological materials to the scaffold. For example, the scaffold construct may be coated with a substance that enhances the binding of a cell (e.g. Type I collagen).

A scaffold construct of the present invention may be biodegradable. Biodegradability may be advantageous in applications where the constructs are used as implants. In such cases, biodegradation of the constructs over time may leave re-modelled layer(s) of cells or other structures (e.g. vessels, organs, or components thereof). Biodegradation may be accomplished, for example, by synthesizing polymers with hydrolytically unstable linkages in the backbone (e.g. esters, anhydrides, orthoesters, amides and the like). Additionally or alternatively, constructs of the present invention may be synthesised with materials that are biodegradable upon application in a given biological setting (e.g. implantation in vivo).

Scaffold constructs of the present invention may be used in any suitable application.

The constructs may be used for applications including, but not limited to, cell growth, reproduction and/or differentiation, tissue engineering, and/or medical device applications.

For example, the scaffold constructs may be used as a substrate suitable for supporting cell selection, cell growth, cell propagation and differentiation in vitro as well as in vivo. The scaffold constructs may be used to mimic microenvironments in vivo and thus provide information on cell function.

Additionally or alternatively, the scaffold constructs may be used as biocompatible implants for guided tissue regeneration or tissue engineering.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

The invention will now be described with reference to specific examples, which should not be construed as in any way limiting.

### Example 1

### Preparation of Three-Dimensional Microstructured Tissue Scaffold with Encapsulated Cells by Two-Photon Laser Scanning Photolithography

### Materials and Methods

Preparation of Crosslinkable Fibrinogen Mixture

A photochemical cross-linking method was used to polymerize fibrinogen (see, method described in Elvin et al. (2004), “The development of photochemically crosslinked native fibrinogen as a rapidly formed and mechanically strong surgical tissue sealant”, Biomaterials, 25:2047-5). 15 mg of fibrinogen powder (bovine, Type 1-S; Sigma Aldrich) was weighed in a tube. Sodium persulfate (SPS) (Sigma Aldrich) was freshly prepared as a stock solution of 0.5 M in PBS. The photoinitiator, [Rull(bpy)₃]²⁺ (Sigma Aldrich), was prepared as a stock solution of 50 mM in tissue culture grade water. 2 μl of SPS from the stock solution was then diluted to a final working solution of 10 mM by adding 100 μl of PBS solution. The entire volume of the working solution was added to 15 mg of fibrinogen powder, giving a final fibrinogen solution concentration of 150 mg/ml. The mixture was vortexed until the fibrinogen powder had dissolved completely in the diluted SPS solution. The mixture was centrifuged, and 2 μl of [Rull(bpy)₃]²⁺ was added just prior to the polymerization. Alternatively, the mixture without the addition of [Rull(bpy)₃]²⁺ was kept in the dark before the commencement of the experiment.

Degradation of Crosslinkable Fibrinogen

Fibrinogen mixture with a concentration of 150 mg/ml of fibrinogen was prepared in bulk and dispensed as 20-μl aliquots into Eppendorf tubes. They were left to be polymerized by visible light for ˜5 min at room temperature. Human plasmin (Sigma Aldrich) dissolved in tris(hydroxymethyl)aminomethane (Tris)-buffered saline (pH 7.4) as a 500 μg/ml stock solution was diluted to four different concentrations: 0.1, 1.0, 10 and 50 μg/ml. 500 μl of plasmin solutions of different concentrations was added to separate tubes containing 20 μl of photochemically cross-linked fibrinogen. Controls were prepared whereby 500 ml of Tris-buffered saline (instead of plasmin) was added to a tube with 20 μl of cross-linked fibrin. All samples were incubated at 37° C. in a humidified, 5% CO₂ atmosphere. The supernatant of each sample was pipetted out after 24 h, and the protein concentration was measured with a Nanodrop 2000/2000C (ThermoScientific). Measurements were obtained daily over a period of 24 days.

Preparation of Cells Suspended in Fibrinogen Mixture

Trypsinized HUVECs were centrifuged at 800 rμm for 1 min. The supernatant was removed, leaving only 50 μl, which was required to resuspend the cells. The resuspended cell suspension, which contained a high density of cells, was added in the dark to 100 μl of fibrinogen mixture (150 mg/ml of fibrinogen). 2 μl of [Rull(bpy)₃]²⁺ was added to the fibrinogen mixture in the dark just before polymerization by TPLSP.

Patterning of 3D Cell-Encapsulated Scaffolds by TPLSP

A droplet of 8 μl of fibrinogen mixture that contained HUVECs was placed under a microscope (Olympus X61) for TPLSP. The desired structure was designed using Solidworks, and generated in a stereolithography system with a galvanometric mirror scanner (Scanlabs, Munich, Germany). Axial control of the scanned structures was provided by a high-resolution elevation stage (Newport, Irvine, Calif., USA) that stepped with each slice of exposure. Localized polymerization would occur on the laser spot. The structures were built layer-by-layer through a laser scanning process. The device was developed for 5 min in cell culture media.

Cell Culture of HUVECs

HUVECs (CRL-2873™) thawed from cryopreservation was cultured in Endogro Supplement medium kit (Millipore) supplemented with 1% penicillin streptomycin. Cells were recovered from tissue culture dishes/T25 flask with 0.05% trypsin-ethylenediaminetetraacetic acid (EDTA) in PBS. The cells were routinely passaged at 1/5 confluency. All cells were incubated at 37° C. in a humidified, 5% CO₂ atmosphere.

Live/Dead® Assays and Immunostaining

Live/Dead® assay kit (Invitrogen) was used to demonstrate the viability of HUVECs. Live cells are stained green, and dead cells are stained red.

### Results

The methods described above provide an effective method to produce 3D microstructured scaffolds encapsulating HUVECs in a one-step process. Firstly, the fibrinogen mixture was prepared, followed by the addition of HUVECs at a high cell density. The cells were suspended in the fibrinogen mixture, and 7 ml of this cell mixture was added to a cover slip as a droplet. The cover slip with the droplet was placed on a rectangular glass substrate. Two spacers with a thickness of 500 μm were placed onto the edges of the rectangular glass substrate such that when a top glass substrate was placed over the droplet, the height of the mixture was controlled at 500 μm. This “sandwich” configuration of the cell-fibrinogen mixture was then taken to the laser platform for scanning.

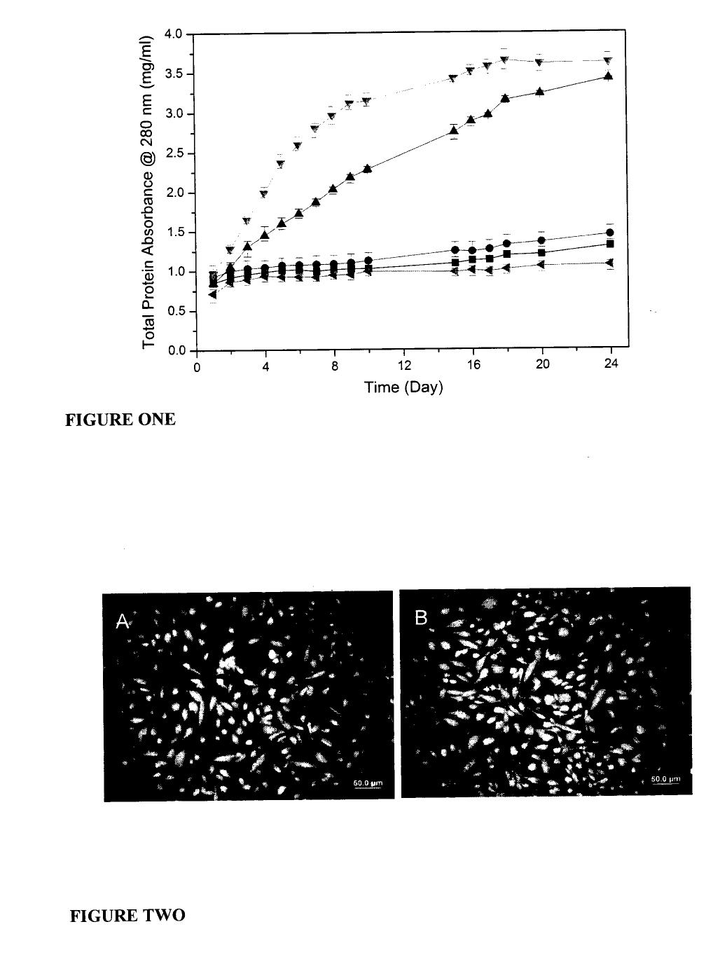

The biodegradation study that was conducted on cross-linked fibrin without the addition of cells demonstrated the ability of cross-linked fibrin to be digested by human plasmin. FIG. 1 shows the degradation of fibrin under different plasmin concentrations over a span of 24 days. The control was set up to test for fibrin's susceptibility to non-enzymatic hydrolysis in the buffer solution. FIG. 1 indicates that lower concentrations of plasmin (0.1 and 1.0 μg/ml resulted in degradation profiles close to that of the control. In contrast, higher concentrations of plasmin (10 and 50 μg/ml) degraded fibrin enzymatically, since their total protein absorbance deviated substantially from that of the control.

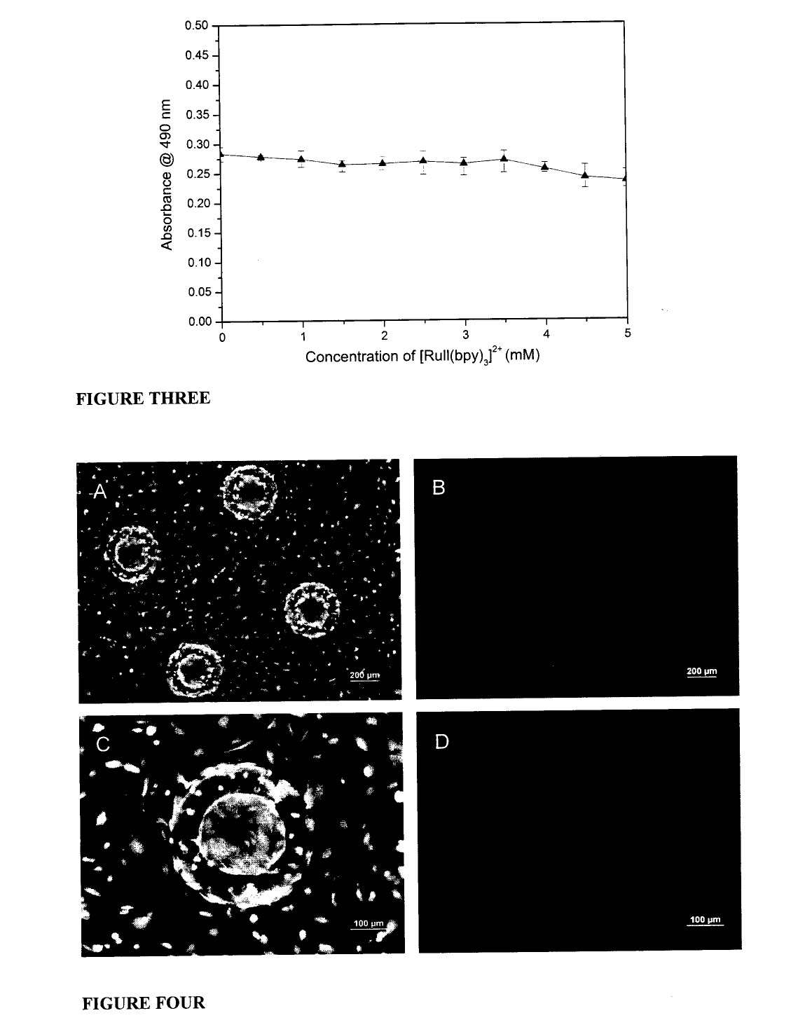

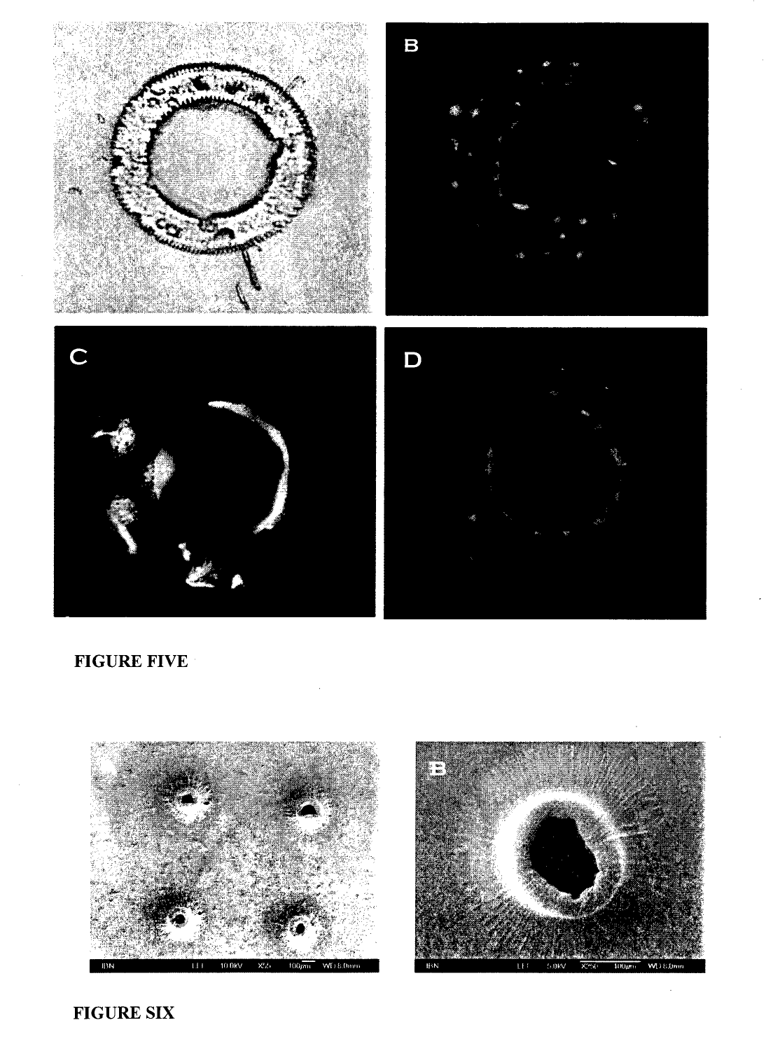

Use of the Live/Dead® assay demonstrated the viability of HUVECs seeded onto the surface of cross-linked fibrin (FIG. 2). Live HUVECs were seen to have attached to and proliferated on the fibrin surface after 48 h, and an insignificant portion of cells were dead. The study of cytotoxicity of [Rull(bpy)₃]²⁺ on HUVECs (FIG. 3) provided additional information on the safe range of [Rull(bpy)₃]²⁺ concentrations (0.5-3.5 mM) to be applied to cross-link the fibrin structures. Typically, 1 mM was used in the cross-linking process. A higher [Rull(bpy)₃]²⁺ concentration would reduce the viability of HUVECs, as reflected by the lower absorbance at 490 nm.

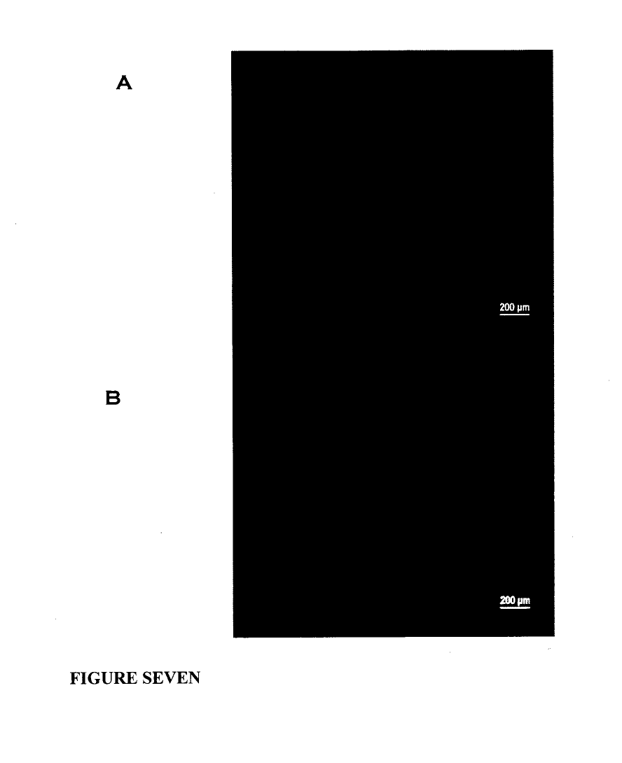

The bright-field images showed the fibrin construct as a solid ring with a slight shadow, illustrating its 3D structures (FIG. 5(A)). The laser beam scanned the fibrinogen mixture as indicated by the lines denoted. The fibrin constructs were freeze-dried for 24 h. Scanning electron microscopy (SEM) images confirmed that the freeze-dried fibrin structures was 3D (see FIG. 6). Confocal microscopy images (with Live/Dead® assay) also substantiated that live cells grew in the 3D microstructured environment. The height of the structure was ˜100 μm, as estimated from the SEM and confocal microscopy images.

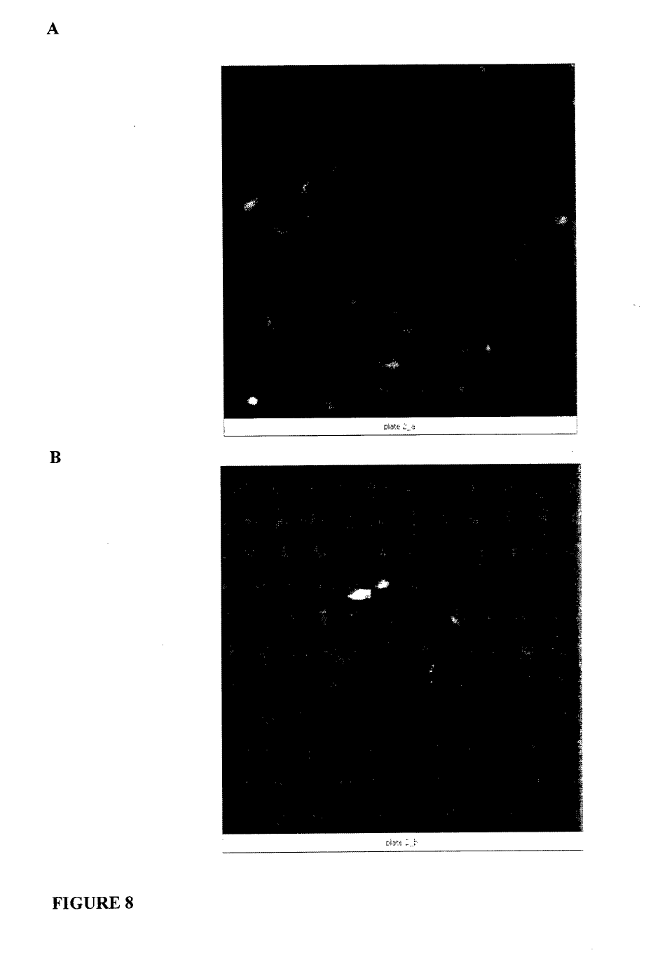

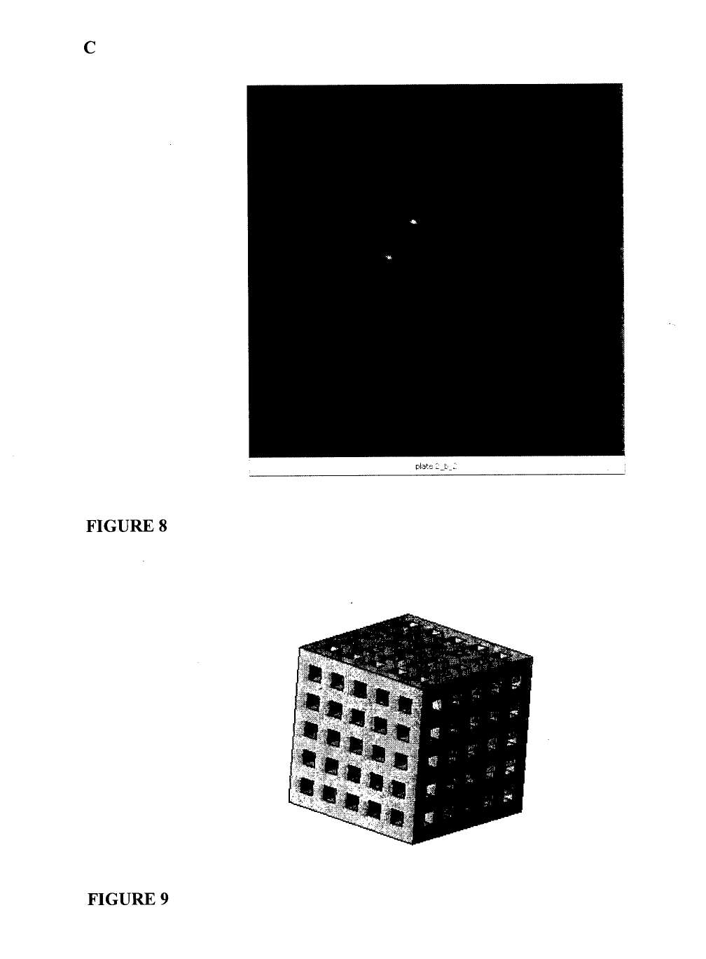

Live/Dead Assay® was employed to verify the viability of the cells grown in the fibrin constructs. HUVECs after 24 h of culture in the fibrin were found to experience fast cell attachment and spreading on the boundaries of the constructs. FIG. 5(C) illustrates that one of the cells elongated along the inner ring of the scaffold after 24 h of culture. HUVECs encapsulated within the 3D fibrin constructs remained viable after 5 days. FIG. 5(D) shows the fluorescent images (with Live/Dead® assay) taken at a certain z-plane in attempt to focus on the cells that proliferated in a 3D manner. Confocal microscopy images validated that cells that were observed to be spreading around the construct grew and stacked over one another. 46 slices of the construct were taken along the z-plane and stacked together (FIG. 8), illustrating that HUVECs were indeed growing along the curvature of the scaffold in a 3D manner.

### Discussion

By providing a favorable microenvironment for the culture of HUVECs, these experiments have demonstrated the value of TPLSP for the fabrication of 3D microstructured scaffolds. The cell microenvironment has a significant influence on cell function and a 3D microenvironment better mimics the physiological environment than does a two-dimensional (2D) cell culture.

As described herein, a platform was developed that facilitated cellular micropatterning by allowing for fast cell attachment onto the scaffold, and hence reducing the time needed for subsequent implantation in various tissue engineering applications. The platform may be used to examine the effect of scaffold geometry on individual cells and cell-cell interactions, and to construct cellular arrays for high-throughput diagnostics.

Cells were encapsulated in these fibrin gels at a high cell density, and were spatially distributed in the final fibrin construct according to the concentration of fibrinogen used. Conventional cell seeding was not necessary using the methods of the invention, thus eliminating the problems associated with that process. The composition of the fibrinogen mixture was easily altered to trap cells homogeneously within the fibrin construct. In the present experiments, the 3D device that encapsulated a homogeneous ring of HUVECs was immersed in the culture media. Hence, HUVECs were considerably well-perfused with the vital nutrients and growth factors that would ensure their healthy growth. Mass transfer of nutrients was especially efficient when the construct was small (with a diameter of ˜400 μm) relative to the amount of surrounding media. The thickness of the ring structure was <100 μm, which compared favorably to the diffusion limit of 200 μm (from blood vessels) (see Botchwey E A, et al. (2003), “Tissue engineered bone: Measurement of nutrient transport in three-dimensional matrices”, J Biomed Mater Res; 67A:357-67). It thus facilitated passive diffusion of nutrients from the culture media across the thin porous walls of the fibrin structure to the cells, and allowed for cell attachment and proliferation within the fibrin structure (FIG. 5). HUVECs were seen to elongate within the fibrin and grow to form confluent layers of cells. It was evident from the confocal microscopy images that the cells adhered within the fibrin structure in a 3D manner and were aligned along the curvature of the device. In fact, the cells were able to attach themselves onto the curvature of the fibrin structure as rapidly as after 1 day of culture. Fast cell attachment illustrated that the fibrin structure generated is not only cell-adhesive, but also able to direct the way in which the cells would grow.

These 3D fibrin constructs can also act as functional units to better mimic the microenvironment in order to conduct advanced studies on cell function and processes, such as cell proliferation and death. HUVECs that piled up along the boundaries of the fibrin constructs exhibited the ability of the 3D scaffolds to accommodate a stack of cells (with a dimension of ˜10 μm each) up to a height of 100 μm.

The use of [Rull(bpy)₃]²⁺ led to cross-linked fibrin products with a very high yield.

This was because [Rull(bpy)₃]²⁺ is a strong light-harvesting molecule that would provide rapid and effective protein cross-linking in the presence of visible light Infrared was used in TPLSP to photo-excite [Rull(bpy)₃]²⁺ in our experiment. Polymerization of the constructs was evident from the bright-field image of the structures after the washing of the unpolymerized fibrinogen mixture. The constructs maintained their structure after 5 days of culture as shown in FIG. 4 and FIG. 5. Live/Dead® assay demonstrated that the cells were not affected by the infrared irradiation. FIG. 4 illustrates that HUVECs within and along the boundaries of the device were stained green, denoting the viability of the cells. A few red spots were observed, which were thought to be dead cells stained by the ethidium homodimer-1 (EthD-1) dye. However, when only the EthD-1 dye from the Live/Dead® assay kit was added to a fibrin construct without cell encapsulation, the entire construct was stained red as shown in FIG. 7. This indicated that the fibrin construct absorbed the red dye, producing an auto-fluorescence.

An in vitro biodegradation study was conducted on the fibrin device. Human plasmin was used in this study since it is extensively available in our blood stream upon activation. Hence, our device with a confluent layer of endothelial cells would likely respond to the enzymatic action of plasmin following implantation. FIG. 1 shows that 50 μg/ml of plasmin degraded the cross-linked fibrin effectively. Since the physiological concentration of plasmin ranges from 100 to 200 μg/ml (see Becker, (1997), “Textbook of coronary thrombosis and thrombolysis”, Kluwer Academic Publishers; 4:53-55), the device is degradable when used as an implant leaving behind the remodelled layer(s) of endothelial cells.

As cell functions could be better demonstrated by 3D versus 2D cell cultures, the constructs present a useful tool for studying cancer-causing cells and their associated signaling pathways. The approach utilised also provides for the fabrication of tissue-engineered scaffolds with the desired biodegradability, cell compatibility, and ability to promote 3D cell proliferation. Furthermore, since more complex structures could be readily derived with the TPLSP, the methods can be used for the construction of an array of hierarchical structures with the necessary extracellular matrix/fibronectin, which would better mimic the cellular microenvironment.

In summary, the experiments demonstrate the use of TPLSP for the fabrication of fibrin scaffolds. 3D microstructured scaffolds were derived with submicron resolution with high reproducibility and at a good speed, based on a digitized model. The fibrin constructs were fabricated in a manner that enabled entrapment of cells at high density and viability. The scaffolds provided for mechanical support and directed cell spreading according to the shape and curvature of the constructs. Fibrin was found to be biodegradable, non-toxic and cell-compatible. 3D constructs of complex structures could be achieved by this approach to mimic appropriate microenvironments for studying cell functions and conduct basic biological studies, such as cell-cell interactions.

### Example 2

### Preparation of Three-Dimensional Microstructured Tissue Scaffold with for Cell Seeding by Two-Photon Laser Scanning Photolithography

### Materials and Methods

Fabrication of 3D Scaffolds by TPLSP

The photocurable polymer (Accura™ SI10) was obtained from 3D Systems (Rock Hill, S.C., USA). The desired scaffold was designed using CAD software (FIG. 9), and generated in a stereolithography system with a galvanometric mirror scanner (Scanlabs, Munich, Germany). An isolator was placed in front of the laser aperture to prevent reflected laser light from returning to the laser cavity. An acousto-optic modulator (AOM) served as a high-speed shutter for the system. The beam expander (Scanlabs, Munich, Germany) acted as the on-the-fly focusing module to automatically correct for any plane distortion. Axial control of the scanned structures was provided by a high-resolution elevation stage (Newport, Irvine, Calif., USA) that stepped with each slice of exposure. Localized polymerization would occur on the laser spot. The structures were built layer-by-layer through a laser scanning process. The device was developed for 1 h in acetone and rinsed with isopropanol. UV-vis spectra of polymerized and non-polymerized samples were acquired on an Agilent 8453 UV-Visible Spectrophotometer (Santa Clara, Calif., USA).

Primary Rat Hepatocyte Isolation and Cell Culture

Primary hepatocytes were harvested from 7-8 week old male Wistar rats weighing 250-300 g by a two-step in situ collagenase perfusion method. The animals were handled according to the IACUC protocol. Viability of the hepatocytes was determined to be >90% by Trypan Blue exclusion assay (Invitrogen, Carlsbad, Calif., USA). Freshly isolated hepatocytes were seeded onto collagen-coated substrates at a density of 2×10⁵ cells/cm² in a 24-well plate (3.5×10⁵ cells/well), and cultured in Hepatozyme (Invitrogen, Carlsbad, Calif., USA) supplemented with 0.1 μM of dexamethasone (Sigma, St. Louis, Mo., USA), 100 units/ml of penicillin and 100 μg/ml of streptomycin (Invitrogen, Carlsbad, Calif., USA). Cells were incubated with 5% of CO₂ at 37° C. and 95% humidity for 24 h.

For the hepatocyte culture, 3D scaffolds were fabricated as a cube of 2.5 mm×2.5 mm×2.5 mm with a pitch size of 250 μm, and coated with Type I collagen. A 40-μm Nylon Cell Strainer membrane (B D Falcon, San Jose, Calif., USA) was glued (Dow Corning, Midland, Mich., USA) to 5 sides of the cube to create a capillary force to encapsulate the hepatocytes homogeneously in the scaffold, as well as to allow medium and waste exchange. 4×10⁶ hepatocytes were seeded onto the 3D scaffold via the uncovered side of the cube. The cell-seeded scaffold was then placed on a rotator (Biosan Laboratories, Warren, Mich., USA) in an incubator overnight to enhance homogeneous cell seeding.

To prepare a monolayer control for the hepatocyte culture experiment, 2D polymeric substrates were prepared by coating a photopolymer (Accura™ SI10) on Nunc treated 24-well cell culture plates (Thermo Fisher Scientific, Waltham, Mass., USA). The monomers were polymerized with a 600-W UV irradiator (Newport, Irvine, Calif., USA) for 30 min. 70% ethanol and isopropanol were used overnight to sterilize the coated polymer and to remove photochemical waste, respectively. Each substrate was washed at least three times with 1000 μl of 1× phosphate buffered saline (PBS). 200 μl of 1.5 mg/ml of Type I collagen were coated on the polymer for 4 h before aspiration. 4×10⁶ hepatocytes were seeded onto each 2D polymeric substrate, and the plates were placed in the incubator for further culture.

To assess the viability and distribution of cells seeded on the scaffold, HepG2, a liver cancer cell line with green fluorescence protein (GFP), was seeded on the scaffold. The scaffold was transplanted to a cell culture plate after 4 h of cell seeding, and cultured for 7 days in Dulbecco's modified eagle medium (DMEM) supplemented with 10% of fetal bovine serum (FBS) and 1% of penicillin-streptomycin (PS). HepG2 morphology was observed under a LSM 5 DUO inverted confocal microscope (Zeiss, Jena, Germany). Cell viability was determined qualitatively using a fluorescence microscope (Olympus, IX71) by emission of green fluorescence at an excitation wavelength of 395 nm. Stereo projection was observed slice by slice at steps of 20 μm for 64 slices in total, using the LSM 5 DUO inverted confocal microscope.

Assays of Liver-Specific Function

1 mL and 4 mL of Hepatozyme were collected for the quantification of albumin levels in 2D culture and 3D scaffold, respectively. 500 μl and 4 mL of 5 mM of NH₄Cl were added to each well of the 2D culture and 3D scaffold, respectively, and incubated for 90 min for the urea assay.

Culture medium was assayed for albumin and urea secretion. The albumin production of hepatocytes was measured every 24 h using the rat albumin enzyme-linked immunosorbent assay (ELISA) quantitation kit (Bethyl Laboratories, Inc., Montgomery, Tex., US). The urea level of hepatocytes incubated with 5 mM of NH₄Cl was measured using the urea nitrogen kit (Stanbio Laboratory, Boerne, Tex., US). Albumin absorbance and urea absorbance were measured at 450 nm and 520 nm, respectively, with a microplate reader (Tecan Safire, Mannedorf, Switzerland). Concentration values were normalized against the nutrient medium volume and the number of seeded cells.

Immunofluorescence was used to qualitatively demonstrate hepatocyte viability and function. DAPI (Invitrogen, Carlsbad, Calif., USA), Texas Red (Invitrogen, Carlsbad, Calif., USA) and FITC (Abeam, Cambridge, Mass., USA) were used to stain the nuclei, fibronectin and albumin of the hepatocytes. Image J (National Institute of Health, USA) was used to superimpose the images.

Statistics and Data Analysis

All data were presented as mean±standard error of the mean (SEM). Statistical significance was evaluated using the t-test, with the significance level set at p<0.05.

### Results

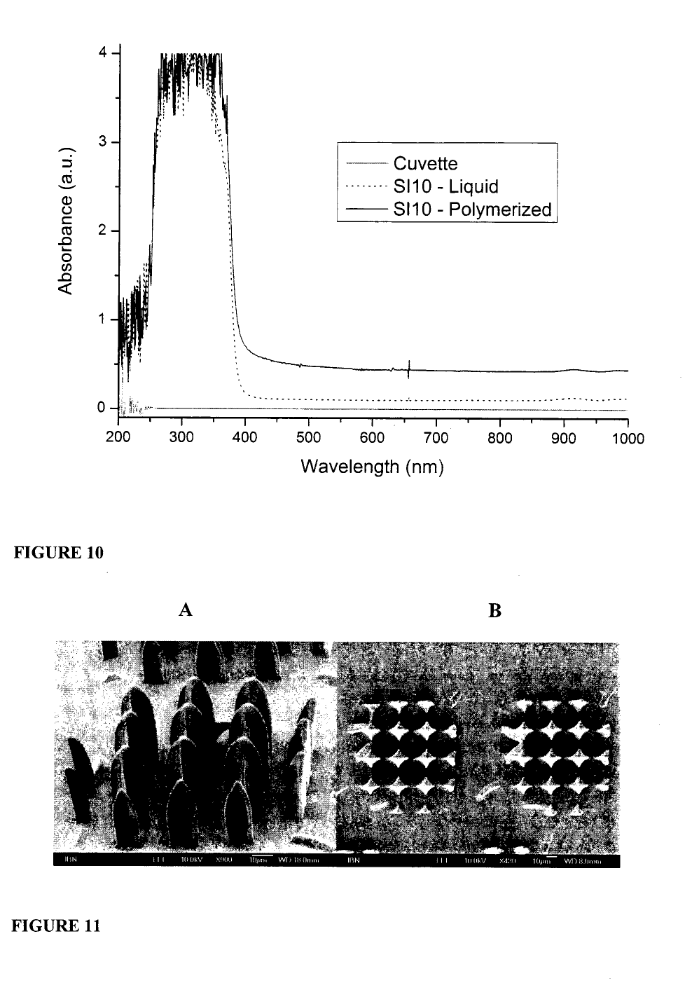

The SI10 photopolymer was characterized by UV-vis spectroscopy (FIG. 10). Absorbance of the liquid monomer in the visible wavelength (400-700 nm) was negligible with reference to the control (an empty cuvette). After polymerization, the absorbance of the solid monomer was still negligible, rendering the entire device almost transparent and easily observed with a fluorescence microscope.

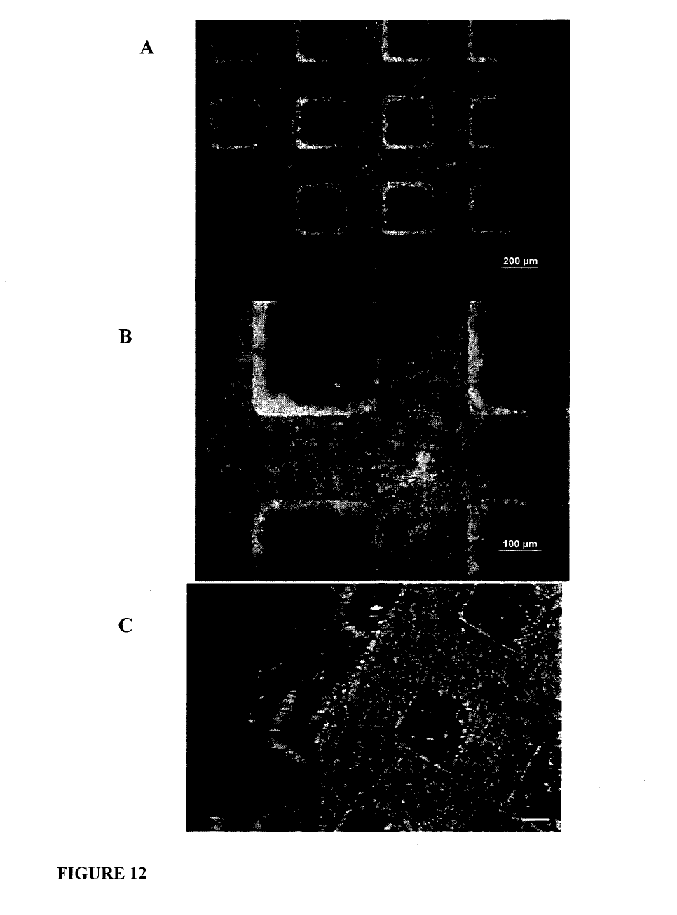

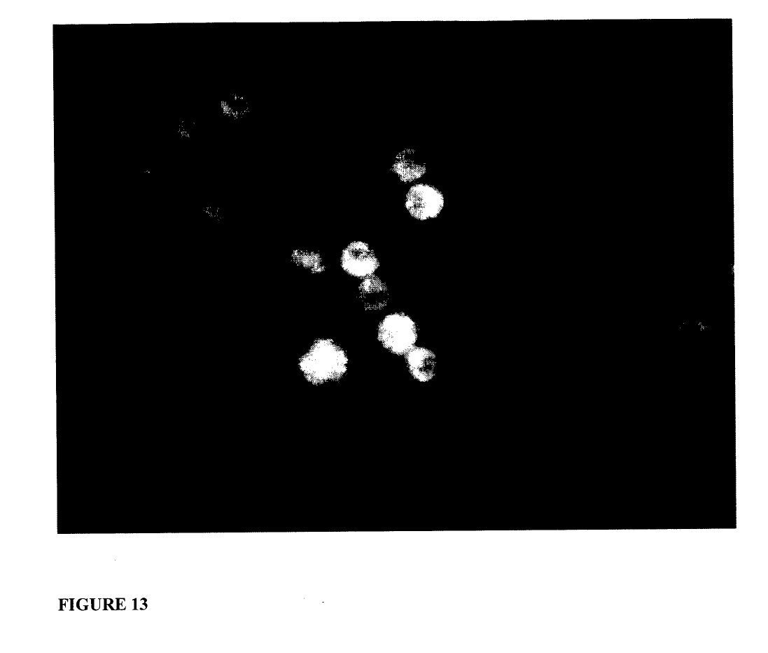

The TPLSP system demonstrated excellent fabrication of microstructures with feature resolution in the micron or submicron range (see example in FIG. 11). The fabrication time for the 2.5 mm×2.5 mm×2.5 mm cubic scaffold depicted in FIG. 1 took only ˜2 h. HepG2 cells attached and proliferated well on the surface of the 3D scaffold. Cells were distributed according to the topography of the structure (FIG. 12). Stereo projection of the confocal images showed homogeneous cell distribution within the 3D scaffold (data not shown).

Primary hepatocytes cultured on the 3D microstructured scaffolds were shown to be viable and functioning on Day 4 of culture as determined qualitatively by immunofluorescence staining, where albumin and fibronectin were shown to be expressed (FIG. 13). For a more quantitative measure of liver-specific function, the supernatant albumin and urea concentrations of primary hepatocyte cultures for both the 3D scaffolds and 2D polymeric substrate controls were used as surrogate markers for the level of protein synthesis and nitrogen metabolism, respectively.

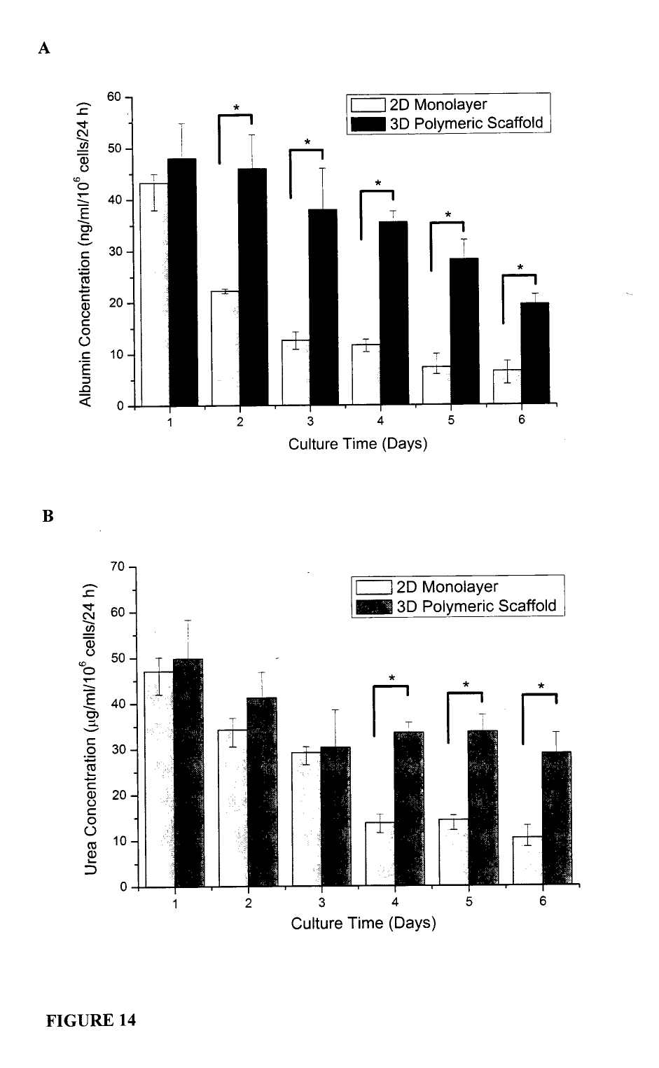

Similar initial levels of albumin and urea on day 1 among the experimental sets indicated that the hepatocytes started off on an equal footing with respect to function (FIG. 14). As the experiment progressed (for Days 2-6 and Days 4-6, respectively), however, the levels of albumin and urea became significantly lower for the 2D substrate as compared to the 3D microstructured scaffold (p<0.05).

### Discussion

2-photon polymerization was first demonstrated by Kawata et al. in 1997 (see Maruo et al. (1997), “Three-dimensional microfabrication with two-photon-absorbed photopolymerization”, Opt Lett; 22:132-4). A clear advantage of 2-photon polymerization as compared to the 1-photon case is the ability for volume polymerization. This has enabled the fabrication of various 3D objects, which have quickly found applications in the areas of exotic optical structures and nano electromechanical systems (NEMS). So far, however, 2-photon photolithography has not been directly applied to scaffold-based tissue engineering due to certain drawbacks and technological limitations of the existing systems.

Certain modifications were made to the system described in these experiments to realize its potential for fabricating biomedical devices and tissue engineering scaffolds. Firstly, in contrast to the use of oil lens in existing devices, the system described herein used an air lens, which avoids the possibility of oil contaminating the sample and the system. Secondly, the oil droplet in the existing devices also places a limit on the scan height of the device (˜1 mm+focal length of the objective), whereas the system described herein allows for a scan height of 30 mm, leading to a greater scan volume. While the scan resolution of our 2D photon device is comparable to existing systems (100 nm), the scan speed (30 mm/s) of the present system is superior to those reported in literature. Having achieved a system performance that provides for practical fabrication of tissue engineering scaffolds, relevant 3D structures of various designs have been attained (FIG. 11).

This study was aimed at demonstrating the utility of a miniaturized 3D structure fabricated by 2-photon photolithography as a tissue engineering scaffold. One focus of the tissue engineering efforts was to engineer liver tissue with functionality that would be useful as a therapy for end-stage liver disease or liver failure. With the final goal of mimicking the layered architecture and interconnectivity of hepatocytes as observed in vivo, a simplified, miniaturized scaffold as a starting point was decided upon. As proof-of-principle, a cubic microstructured 3D scaffold for hepatocyte culture was designed to investigate whether these scaffolds could provide anchorage to primary hepatocytes, while maintaining their differentiated liver-specific function. The 3D cubic scaffold was evaluated in comparison to hepatocyte monoculture on a 2D substrate composed of the same polymer.

As hepatocytes are anchorage-dependent cells, it was important to ensure good cell adhesion as a prerequisite to functionality. Although the polymer itself supported cell attachment (data not shown), both 3D and 2D substrates were coated with collagen Type I to further enhance cell adhesion. To seed the hepatocytes, a Nylon cell filtration membrane was used to seal all sides of the cubic scaffold but one, through which the cells were introduced. Overnight rotation ensured that cells could settle and attach to all the inner surfaces of the scaffold. The effectiveness of the collagen coating as well as cell-seeding procedures was demonstrated by the uniformity of HepG2 cell distribution in the 3D scaffold (FIG. 12). Following that, primary hepatocytes were cultured within the scaffolds. Having established viability and function of the cells qualitatively by immunofluorescence on Day 4 of culture (FIG. 13), a further set of cultures was subjected to albumin and urea assays to provide a quantitative measure of liver-specific function over 6 days.

In the 2D monoculture controls, there was a significant drop in functionality of hepatocytes from Day 1 to Day 2. Monolayer culture is favored in the industrial setting due to the high efficiency of nutrient transport by the medium. However, the absence of appropriate microenvironmental architecture, leading to the lack of cell-cell communication, appeared to be detrimental to hepatocyte function. In contrast, for the case of the 3D polymeric scaffolds, there was only a slight decrease in albumin and urea levels from Day 1 to Day 2, and the urea level was stabilized from Day 3 onwards (FIG. 14). By providing the right microenvironmental architecture to the cells, the 3D scaffold had helped to maintain the functionality of cells, while still providing for efficient nutrient transport. For both 3D and 2D culture, the reduction in albumin and urea levels between Day 1 and Day 2 could be due to unattached hepatocytes that were not completely removed by washing after overnight seeding, thus contributing to the slightly higher levels on the first day.

The higher functionality of the hepatocytes cultured in the 3D scaffold as compared to monoculture could be due to the presence of good homotypic cell-cell contact or the higher volume density of hepatocytes within the scaffold, which led to higher local concentrations of soluble factors that were important for maintaining the hepatocyte phenotype. As the seeding density of hepatocytes for both the 3D scaffold and monolayer was high and above the threshold reported to promote cell-cell interaction and therefore liver-specific function, the difference in function could be attributed to the effect of soluble factors rather than cell-cell interaction.

This work has demonstrated the value of TPLSP for the fabrication of 3D microstructured scaffolds, which provide a favorable microenvironment for the culture of cells, as exemplified by the maintenance of liver cell function. It also underlines the need to fabricate elaborate, well-defined scaffolds for functional tissue engineering. Conventional lithography on a silicon chip is not suitable due to material incompatibility and the complexity of 3D fabrication. In contrast, TPLSP offers a convenient method by which arbitrary physical scaffolds can be printed slice-by-slice according to a digitized drawing. Therefore, the range of potential microstructures is limited only by imagination and rational design. While a commercially available photo-curable polymer has been employed for this study, other potentially more suitable polymers may be used to fabricate scaffolds with the same degree of resolution and fidelity. These include bioresorbable polymers, and/or polymers with pendant functional groups that are either biologically active or could be used to tether biologically active molecules such as growth factors.

The present study developed TPLSP as a method for the fabrication of 3D microstructured scaffolds. Scaffolds can be fabricated with submicron resolution with high reproducibility and at a good speed, based on a digitized model. Primary hepatocytes cultured within a cubic microstructured scaffold maintained higher liver-specific functions over a period of 6 days, superior to hepatocytes cultured in a monolayer, demonstrating the advantage of TPLSP-fabricated 3D scaffolds for tissue engineering.

1. A method for producing a three-dimensional scaffold construct comprising encapsulated cells, the method comprising:
(a) providing a solution comprising cells to be encapsulated, a photoinitiator, and a plurality of units capable of forming polymer chains;
(b) providing a photolithography instrument comprising a two-photon laser;
(c) using the instrument to apply the laser to the solution to activate the photoinitiator thereby facilitating polymerisation of said units to form polymer chains, and, cross-linking of the polymer chains;
wherein the laser is applied to the solution in three-dimensions in a pre-defined pattern to assemble said construct, and said cells are encapsulated within the assembled construct; and
(d) culturing the construct of (c) comprising the encapsulated cells.

2. The method according to claim 1, wherein the scaffold construct is assembled according to a three dimensional computer assisted design (CAD) image that is read by said photolithography instrument.

3. The method according to claim 1, wherein the laser emits energy in the infrared region.

4. The method according to claim 1, wherein the cells comprise human umbilical vascular endothelial cells (HUVEC).

5. The method according to claim 1, wherein the cells comprise hepatocytes.

6. The method according to claim 1, wherein the cells comprise stem cells.

7. The method according to claim 1, wherein the construct comprises more than one type of polymer chain.

8. The method according to claim 1, wherein the unit is monomer of a resin polymer.

9. The method according to claim 1, wherein the unit is a fibrillar protein.

10. The method according to claim 9, wherein the fibrillar protein is fibrinogen.

11. The method according to claim 10, wherein the photoinitiator is ruthenium II trisbipyridyl chloride [Rull(bpy)₃]²⁺, and the solution comprises an oxidising agent.

12. The method according to claim 11, wherein the oxidising agent is sodium persulfate.

13. The method according to claim 1, wherein the construct is ring-shaped.

14. The method according to claim 1, wherein the pores are between about 1 μm and about 10 μm in width or diameter.

15. The method according to claim 1, wherein further comprising washing the construct to substantially remove non-crosslinked polymer chains and non polymerised units.

16. The method according to claim 1, wherein the polymer chains are biodegradable.

17. The method according to claim 1, herein the solution further comprises a bioactive component.

18. The method according to claim 1, wherein the cells are in the solution at a concentration of between about 1×10⁶/ml and about 1×10⁷/ml.

19. The method according to claim 1, further comprising seeding additional cells to the construct after completion of said polymerization and cross-linking.

20. The method according to claim 13, wherein the ring-shaped construct has a diameter of about 400 μm, and a thickness of about 100 μm.
